SEQUENCE LISTING (9 sequences)

SEQ ID NO: 1 - protein, 23 aa, Solanum tuberosum
  misc_feature: StPep1
ATERRGRPPSRPKVGSGPPPQNN

SEQ ID NO: 2 - protein, 23 aa, Solanum lycopersicum
  misc_feature: SlPep6
ATDRRGRPPSRPKVGSGPPPQNN

SEQ ID NO: 3 - protein, 23 aa, Arabidopsis
  misc_feature: AtPep1
ATDRRGRPPSRPKVGSGPPPQNN

SEQ ID NO: 4 - DNA, 55 bp, Artificial Sequence
  analog of StPep1
  misc_feature: StPep1-FP
caccgagctcgcaaccgagagaagaggtagacctcctagccgtcctaaagtcggc

SEQ ID NO: 5 - DNA, 54 bp, Artificial Sequence
  Analog of StPep1
  misc_feature: StPep1-RP-NOSTOP
agcttctagaattgttctgcggcggaggcccggagccgactttaggacggctag

SEQ ID NO: 6 - DNA, 32 bp, Artificial Sequence
  analog of StPep1
  misc_feature: StPep1-RP-STOP
agcttctagattaattgttctgcggcggaggc

SEQ ID NO: 7 - DNA, 93 bp, Artificial Sequence
  Codon optimized AtPep1 sequence
acaaaagtcaaggcgaaacaacggggaaaagaaaaagtgtccagtgggagaccgggacaacataactgatctcggatgcagtctacaatgcag

SEQ ID NO: 8 - DNA, 81 bp, Artificial Sequence
  Codon optimized SlPep6 sequence
gcaaccgacagaagaggtcgtcctccatcacgtccaaaggtggggagcggccctccaccacaaaataattctagaaagatg

SEQ ID NO: 9 - DNA, 66 bp, Artificial Sequence
  Codon optimized SlPep1 sequence
gcaaccgagagaagaggtagacctcctagccgtcctaaagtcggcgggcctccgccgcagaacaat

(19) United States
(12) Patent Application Publication
(10) Pub. No.: US 2021/0355500 A1
(43) Pub. Date: Nov. 18, 2021

(54) BACTERIALLY SECRETED IMMUNOSTIMULANTS AND METHODS TO PROTECT AGAINST PATHOGENS

(71) Applicant: Washington State University, Pullman, WA (US)

(72) Inventors: Cynthia GLEASON, Pullman, WA (US); Lei ZHANG, West Lafayette, IN (US)

(21) Appl. No.: 17/320,414

(22) Filed: May 14, 2021

Related U.S. Application Data
(60) Provisional application No. 63/024,648, filed on May 14, 2020.

(51) Int. Cl. C12N 15/82; A01N 37/46

(52) U.S. Cl. CPC C12N 15/8285; C12R 2001/125; A01N 37/46

(57) ABSTRACT

The present disclosure provides bacterial strains modified for secretion of a plant defense elicitor peptide. More particularly, the bacterial strain comprises an exogenous sequence encoding the plant defense elicitor peptide. Vectors and methods utilizing the described bacterial strains are also provided.

### CROSS-REFERENCE TO RELATED APPLICATIONS

This application claims the benefit under 35 USC § 119(e) of U.S. Provisional Application Ser. No. 63/024,648, filed on May 14, 2020, the entire disclosure of which is incorporated herein by reference.

### GOVERNMENT RIGHTS

This invention was made with government support under Grant/Contract Numbers 2019-67013-29963, awarded by the United State Department of Agriculture, National Institute of Food & Agriculture. The government has certain rights in the invention

### TECHNICAL FIELD

The present disclosure provides bacterial strains modified for secretion of a plant defense elicitor peptide. More particularly, the bacterial strain comprises an exogenous sequence encoding the plant defense elicitor peptide.

### BACKGROUND AND SUMMARY OF THE INVENTION

Root-knot nematodes, generally of the Meloidogyne spp., are major agricultural pests and cause billions of dollars in plant yield losses each year. For example, the temperate root-knot nematode Meloidogyne chitwoodi is endemic to the potato growing regions of the Pacific Northwest of the United States, as well as areas of Europe, Asia, and Africa. This species of root-knot nematode is of importance to production of potatoes and other dicots because its potential to infect the tubers, thus causing unsightly galling and brown spots to form under the skin. Commercially available potato cultivars do not have natural genetic resistance to M. chitwoodi. As such, potato producers must currently rely on costly nematicides in order to manage root-knot nematodes in potato fields. Thus, there exists a need to develop alternative means for controlling root-knot nematodes in plants such as potatoes.

Plants detect tissue damage/injury by wounding or pathogen attack and release endogenous molecules called Damage-Associated Molecular Patterns (DAMPs). An example of DAMPs is known as a plant elicitor peptides (Peps). Although Pep treatments have been shown to trigger both local and systemic defense responses in Arabidopsis thaliana and maize, such effects have not been extensively studied in other crop types such as potato.

Accordingly, the present disclosure provides a novel strategy capable of utilizing Peps to activate or enhance the plant immune system for efficiently controlling root-knot nematodes. The disclosure provides a novel bacterial strain system to deliver plant defense elicitors useful for controlling nematodes, for example, Meloidogyne chitwoodi, Meloidogyne incognita, and the like. Use of the described bacterial strains and related compositions are highly adaptable and novel methodologies that can be utilized in improving dicot plants such as those from the Solanaceae species (e.g., potatoes, tomatoes, Arabidopsis, etc.) as described herein.

The compositions and methods of the present disclosure provide several advantages and improvements compared to the state of the art. First, gene expression analysis indicate that the compositions when utilized to treat potato roots caused local and systemic changes in gene expression, and the global changes to potato root gene expression correlates with providing resistance to root-knot nematode infections. Second, the compositions and methods utilize a unique delivery method of the plant defense elicitors, in particular a non-plant delivery system increase the resistance to root-knot nematodes. Third, the compositions and methods provide an alternative to reliance on costly nematicides in order to manage root-knot nematodes in the field.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIGS. 1A-1F show that StPep1 pre-treatment of potato roots reduces M. chitwoodi galling and egg masses numbers with no adverse effects on plant biomasses. Potatoes were watered with 1 μM StPep1 solution, and 2 days later the plants were inoculated with M. chitwoodi juveniles. The number of galls on potato roots were counted at 12 days post inoculation (DPI) to evaluate the nematode infections. StPep1 pre-treatment significantly reduced the number of galls/plant (FIG. 1A), and galls/gram root (FIG. 1B). FIGS. 1C and 1D show that the StPep1 pre-treatment had no effect on the root or shoot weights of the nematode inoculated plants. The number of egg masses on potato roots were stained with phloxine B and counted at 35 DPI. StPep1 pretreatment significantly reduced the number of egg masses/plant (FIG. 1E) and egg masses/gram root (FIG. 1F). Data shown represents means±SEM; number of plants (n, number of plants)=10, **P<0.01 using two-tailed Student's t-test.

FIGS. 2A-2D show Bacillus subtilis secreting StPep1 reduces M. chitwoodi infections on potato roots. Russet Burbank potato plants were pre-treated with B. subtilis transformed with StPep1-His or StPep1 prior to M. chitwoodi inoculation. B. subtilis transformed with empty vector pBE-S (EV) and a water only treatment was used as controls. Plants watered with B. subtilis transformed with StPep1-His or StPep1 showed significantly reduced numbers of galls per plant (FIG. 2A) and number of galls per gram root (FIG. 2B), compared with those of the controls (FIGS. 2C and 2D), pre-treatments with engineered B. subtilis had no effects on the root or shoot fresh weights of nematode inoculated plants. Data are means±SEM (n=10). Statistical comparisons were done using a one-way analysis of variance (ANOVA), followed by Tukey's multiple-comparison test (set at 5%), and different lowercase letters indicate significant differences between treatments.

FIGS. 3A-3B show the effect of StPep1 treatment on M. chitwoodi invasion of potato roots. Russet Burbank potato plants were pre-treated with StPep1 or mock solution, and 2 days later the plants were inoculated with M. chitwoodi juveniles. The nematodes within roots were visualized by acid fuchsin staining and counted at 4 days post inoculation. StPep1 pre-treatment does not significantly affect the M. chitwoodi invasion of potato roots, in terms of the number of J2/plant (FIG. 3A) and the number of J2/gram root (FIG. 3B). Data shown represents means±SEM (n=10; ns indicates not significant using unpaired Student's t-test).

FIG. 4 shows the effect of StPep1 treatment on M. chitwoodi viability. Second stage juveniles (J2) were incubated for 2, 4, and 7 days in 1 μM of StPep1 or mock solutions, and the percentage of live J2 was calculated. Data shown represents means±SEM (n=10 biological samples). Ns indicates not significant using Student's t-test.

FIGS. 5A-5C show detection of StPep1-His expression and secretion by engineered B. subtilis. FIG. 5A shows Western blot using anti-His tag antibody detected presence of the StPep1-His fusion peptide in the transgenic B. subtilis. FIGS. 5B-5C show detection of StPep1-His secretion in the bacterial supernatant by ELISA. FIG. 5B shows standard curve using a serial dilution of standard with known concentration. FIG. 5C shows absorbance at OD450 of purified His-tag proteins from the supernatant of transgenic B. subtilis culture (n=3 biological replicates). Estimated StPep1-His concentrations are shown.

FIG. 6 shows the quantification of bacterial cell counts of Bacillus subtilis-secreting StPep1 on potato roots at 1, 4, and 12 days post inoculation. Data shown represents means±SEM (n=7).

FIGS. 7A-7B show the StPep1 treatment induced expression of defense marker genes in potato leaves, but not in roots. Russet Burbank (RB) potato tissue culture plants were treated by immersing roots in a StPep1 or water solution for 0, 2, 6 and 12 hours. The expression levels of StPINII, StPR1, StWRKY40, and StOsmotin2 in leaves (FIG. 7A) and roots (FIG. 7B) were analyzed by qRT-PCR. Expression was normalized to the potato housekeeping gene elongation factor 1-alpha (StEF1α), and fold expression was calculated by comparing expression of the genes after StPep1 treatment to that of the mock treatment. Asterisks indicate statistically significant difference in expression levels at the corresponding time points from that of the water control. Data shown represents means±SEM (n=3 biological replicates, *P<0.05 using two-tailed Welch's t-test).

FIG. 8 shows the evaluation of RNA-seq gene expression analysis by qRT-PCR. Expression of 17 genes picked from the RNA-seq data was measured by qRT-PCR. Pearson correlation coefficient analysis was used to determine the correlation between RNA-seq and qRT-PCR results.

FIGS. 9A-9E show gene ontology (GO) enrichment analysis for the differentially expressed genes (DEG' s) in potato roots after StPep1 treatment. GO enrichment analysis of up-regulated (FIGS. 9A-9B) and down-regulated genes (FIGS. 9C-9E) was performed using Fisher's exact test. Representative subsets of enriched GO terms were summarized using REVIGO in terms of molecular functions (MF), biological processes (BP) and cellular components (CC). No enriched CC terms were found for up-regulated genes.

FIGS. 10A-10B show the expression analysis of StPep1-induced marker genes in potato roots and leaves. Expression levels of the 8 selected genes were measured by qRT-PCR in Russet Burbank potato roots (FIG. 10A) and leaves (FIG. 10B) treated with StPep1 or mock control solution by root immersion for 6 hours. Data shows fold change gene expression compared to the control. Asterisks indicate statistically significant difference in expression levels between StPep1 and control treatments. Data shown represents means±SEM (n=3 biological replicates, *P<0.05 and **P<0.01 using two-tailed Welch's t-test).

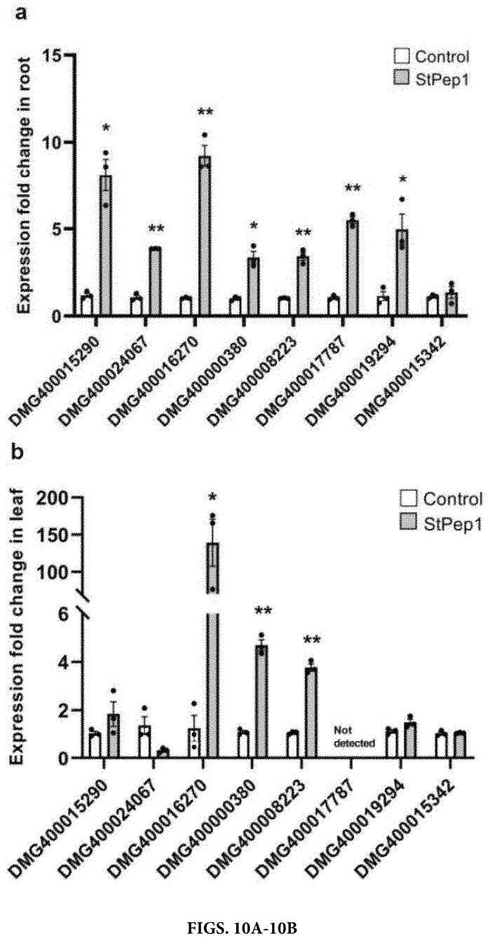

FIG. 11 shows the expression analysis of StPep1-induced marker genes in potato roots after treatment with B. subtilis transformed with StPep1-His. qRT-PCR assessed gene expression levels of the 7 StPep1-induced genes in potato roots. The plant roots were treated with cultures of B. subtilis transformed with StPep1-His, B. subtilis with the empty vector pBE-S (EV), or with water (control) for 6 hours. Data shows fold change gene expression compared to the control. Data shown represents means±SEM (n=3 biological replicates). For each gene, multiple comparisons among control, EV and StPep1-His were carried out using Tukey's multiple-comparison test (set at 5%) and different lowercase letters indicate significant differences between treatments.

FIGS. 12A-12D show the expression analysis of StPep1-induced marker genes in StCOI1-knockdown and NahG-expressing potato plants. FIG. 12A shows StCOI1 transcript levels were detected by qRT-PCR in WT (Desiree) and transgenic Desiree plants carrying the StCOI1-RNAi construct. The potato housekeeping gene StEF1α was used as internal reference. Knockdown plants have reduced StCOI1 expression. FIG. 12B shows qRT-PCR assessed gene expression levels of the 7 StPep1-induced genes in the roots of StCOI1 knockdown plants treated with StPep1 or mock treatment for 6 hours. Expression fold change was calculated as between the StPep1 and control. Asterisks indicate statistically significant difference in fold change of expression between potato transgenic lines and their corresponding WT. Data shown represents means±SEM (n=3 biological replicates, *P<0.05 using two-tailed Welch's t-test). FIG. 12C shows the NahG-expressing line is in the background of Bannock Russet (WT). The presence of transcripts from the NahG gene were tested using reverse transcription-PCR in WT (Bannock Russet) and transgenic plants overexpressing the bacterial NahGgene using NahG-specific primers. StEF1α-specific primers were performed in a control reaction. The NahG-expressing plants were positive for the transgene. FIG. 12D shows qRT-PCR assessed gene expression levels of the 7 StPep1-induced genes in the roots of NahG-expressing plants treated with StPep1 or mock treatment for 6 hours. For qRT-PCRs, expression fold change was calculated as the ratio between the StPep1 and control. Asterisks indicate statistically significant difference in gene expression between potato transgenic lines and their corresponding WT. Data shown represents means±SEM (n=3 biological replicates, *P<0.05 using two-tailed Student's t-test).

### DETAILED DESCRIPTION

In illustrative aspect, a bacterial strain modified for secretion of a plant defense elicitor peptide is provided. The bacterial strain comprises an exogenous sequence encoding the plant defense elicitor peptide.

In an embodiment, the bacterial strain is a Gram-positive bacterial strain. In an embodiment, the bacterial strain is a Gram-negative bacterial strain. In an embodiment, the bacterial strain is a Bacillus bacterial strain. In an embodiment, the Bacillus bacterial strain is a root-associating Bacillus strain. A Bacillus strain can be considered to be a root-associating Bacillus strain when it is located near, on, or adjacent to roots after application to a plant or the area around a plant. In an embodiment, the Bacillus bacterial strain is Bacillus subtilis.

In an embodiment, the bacterial strain is a Escherichia bacterial strain. In an embodiment, the Bacillus bacterial strain is a root-associating Escherichia strain. A Escherichia strain can be considered to be a root-associating Escherichia strain when it is located near, on, or adjacent to roots after application to a plant or the area around a plant. In an embodiment, the Escherichia bacterial strain is Escherichia coli.

In an embodiment, the plant defense elicitor peptide is StPep1. StPep1 is a potato defense elicitor peptide and is identified as SEQ ID NO: 1

(ATERRGRPPSRPKVGSGPPPQNN).

In an embodiment, the plant defense elicitor peptide is SlPep6. SlPep6 is a tomato defense elicitor peptide and is identified as SEQ ID NO: 2

(ATDRRGRPPSRPKVGSGPPPQNN).

In an embodiment, the plant defense elicitor peptide is AtPep 1. AtPep 1 is an Arabidopsis defense elicitor peptide and is identified as SEQ ID NO: 3

(ATKVKAKQRGKEKVSSGRPGQHN).

In an embodiment, the exogenous sequence encoding the plant defense elicitor peptide is codon optimized.

In an embodiment, the exogenous sequence encoding the plant defense elicitor peptide is SEQ ID NO: 1 (ATERRGRPPSRPKVGSGPPPQNN). In an embodiment, the exogenous sequence encoding the plant defense elicitor peptide is a sequence having at least 95% sequence identity to SEQ ID NO: 1.

In an embodiment, the exogenous sequence encoding the plant defense elicitor peptide is SEQ ID NO: 2 (ATDRRGRPPSRPKVGSGPPPQNN). In an embodiment, the exogenous sequence encoding the plant defense elicitor peptide is a sequence having at least 95% sequence identity to SEQ ID NO: 2.

In an embodiment, the exogenous sequence encoding the plant defense elicitor peptide is SEQ ID NO: 3: ATKVKAKQRGKEKVSSGRPGQHN. In an embodiment, the exogenous sequence encoding the plant defense elicitor peptide is a sequence having at least 95% sequence identity to SEQ ID NO: 3.

In an embodiment, the strain is the Bacillus subtilis strain deposited with Agriculture Research Culture Collection (NRRL) and assigned accession number B-68058 with name and strain designation provided as Bacillus subtilis RIK1285; BsStPep1, respectively. In an embodiment, the strain is the Bacillus subtilis strain deposited with NRRL and assigned accession number B-68059 with name and strain designation provided as Bacillus subtilis RIK1285; BsS1Pep6, respectively. In an embodiment, the strain is the Bacillus subtilis strain deposited with NRRL and assigned accession number B-68057 with name and strain designation provided as Bacillus subtilis RIK1285; BsAtPep1, respectively. Each of the three strains assigned accession numbers B-68057, B-68058, and B-68059, were deposited with NRRL on May 28, 2021, located at 1815 North University Street, Peoria, Ill., 61604, U.S.A.

In an embodiment, the modified strain affects the expression of at least 5 marker genes for the plant defense elicitor peptide. In an embodiment, the marker genes for the plant defense elicitor peptide are found in a plant root. In an embodiment, the plant root is a monocot plant root. In an embodiment, the plant root is a dicot plant root. In an embodiment, the plant root is a potato plant root. In an embodiment, the plant root is a tomato plant root. In an embodiment, the plant root is an Arabidopsis plant root.

In an embodiment, the modified strain amplifies innate immunity of a plant. For instance, the modified strain can amplify innate immunity of the plant against a pest such as a nematode. In an embodiment, the plant is a monocot plant. In an embodiment, the plant is a dicot plant. In an embodiment, the plant is a potato plant. In an embodiment, the plant is a tomato plant. In an embodiment, the plant is an Arabidopsis plant.

In an embodiment, the modified strain is configured to colonize one or more roots of a plant for at least 7 days. In an embodiment, the modified strain is configured to colonize one or more roots of a plant for at least 10 days. In an embodiment, the modified strain is configured to colonize one or more roots of a plant for at least 12 days. In an embodiment, the modified strain is configured to colonize one or more roots of a plant for at least 14 days. In an embodiment, the modified strain is configured to colonize one or more roots of a plant for at least 21 days. In an embodiment, the modified strain is configured to colonize one or more roots of a plant for at least 28 days. In an embodiment, the modified strain is configured to colonize one or more roots of a plant for at least 30 days. In an embodiment, the plant is a monocot plant. In an embodiment, the plant is a dicot plant. In an embodiment, the plant is a potato plant. In an embodiment, the plant is a tomato plant. In an embodiment, the plant is an Arabidopsis plant.

In an embodiment, the modified strain is configured to colonize one or more roots of a plant and increase resistance of the plant to a root-knot nematode. In an embodiment, the plant is a monocot plant. In an embodiment, the plant is a dicot plant. In an embodiment, the plant is a potato plant. In an embodiment, the plant is a tomato plant. In an embodiment, the plant is an Arabidopsis plant. In an embodiment, the root-knot nematode is a Meloidogyne nematode. In an embodiment, the Meloidogyne nematode is Meloidogyne chitwoodi, Meloidogyne incognita, or both. In an embodiment, the Meloidogyne nematode is Meloidogyne chitwoodi. In an embodiment, the Meloidogyne nematode is Meloidogyne incognita.

In illustrative aspect, an E. coli or B. subtilis bacterial expression vector is provided. The bacterial expression vector comprises a nucleic acid sequence encoding a plant defense elicitor peptide; a bacterial promoter functional in E. coli and/or B. subtilis, wherein said promoter is operably linked to said defense elicitor peptide encoding sequence; a nucleic acid sequence encoding a secretory signal peptide, wherein said secretory signal peptide encoding sequence is operably linked to said defense elicitor peptide encoding sequence; and a sequence encoding a selectable marker.

In an embodiment, the plant defense elicitor peptide is selected from the group consisting of StPep1 (SEQ ID NO: 1: ATERRGRPPSRPKVGSGPPPQNN), SlPep6 (SEQ ID NO: 2: ATDRRGRPPSRPKVGSGPPPQNN) and AtPep1 (SEQ ID NO: 3: ATKVKAKQRGKEKVSSGRPGQHN).

In an embodiment, the promoter is aprE and the secretory signal peptide is aprE SP. In an embodiment, the bacterial expression vector further comprises 1 to 16 UAS sequences operably linked to said promoter sequence. In an embodiment, said selectable marker is an antibiotic resistance gene product. In an embodiment, the bacterial expression vector further comprises a replication region for E. coli and/or B. subtilis.

In illustrative aspect, a method of controlling root galling in a plant is provided. The method comprises the step of administering a composition comprising a bacterial strain modified for secretion of a plant defense elicitor peptide to the plant, wherein the bacterial strain comprises an exogenous sequence encoding the plant defense elicitor peptide, and wherein administration of the composition to the plant controls root galling in the plant. The previously described embodiments of the bacterial strain modified for secretion of a plant defense elicitor peptide are applicable to the method of controlling root galling in a plant described herein.

In an embodiment, controlling root galling comprises a reduction in root galling disease in the plant. Evaluation or analysis of the control of root galling is well known in the art, for instance by evaluating the average or the number of root galls present on roots of a plant, evaluating the plant yield associated with the control of root galling, evaluating the prevention of root galling in a plant compared to a plant for which no treatment was applied, and the like.

In an embodiment, controlling root galling comprises a reduction in root galling disease phenotype in the plant. In an embodiment, the reduction is a reduction of about 20%. In an embodiment, the reduction is a reduction of about 25%. In an embodiment, the reduction is a reduction of about 30%. In an embodiment, the reduction is a reduction of about 40%. In an embodiment, the reduction is a reduction of about 50%.

In an embodiment, controlling root galling comprises a reduction in gall number in one or more roots of the plant. In an embodiment, the reduction in gall number is a reduction in average number of galls in a population of plants. In an embodiment, the reduction is a reduction of about 20%. In an embodiment, the reduction is a reduction of about 25%. In an embodiment, the reduction is a reduction of about 30%. In an embodiment, the reduction is a reduction of about 40%. In an embodiment, the reduction is a reduction of about 50%.

In an embodiment, the plant is a monocot plant. In an embodiment, the plant is a dicot plant. In an embodiment, the plant is a potato plant. In an embodiment, the plant is a tomato plant. In an embodiment, the plant is an Arabidopsis plant. In an embodiment, the composition comprises a liquid. In an embodiment, the liquid is water. In an embodiment, the administration is applied via irrigation.

In an embodiment, the root galling is caused by a root-knot nematode. In an embodiment, the root-knot nematode is a Meloidogyne nematode. In an embodiment, the Meloidogyne nematode is Meloidogyne chitwoodi, Meloidogyne incognita, or both. In an embodiment, the Meloidogyne nematode is Meloidogyne chitwoodi. In an embodiment, the Meloidogyne nematode is Meloidogyne incognita.

In an embodiment, administering the composition is performed one time per week. In an embodiment, administering the composition is performed one time every 10 days. In an embodiment, administering the composition is performed one time every 14 days. In an embodiment, administering the composition to the plant is performed by treating the soil near or adjacent to the plant. In an embodiment, administering the composition to the plant is performed by treating the soil near or adjacent to one or more roots of the plant. In an embodiment, administering the composition to the plant is performed by treating the plant upon emergence of the plant. In an embodiment, administering the composition to the plant is performed by treating the plant prior to emergence of the plant.

In illustrative aspect, a method of stimulating defense of a plant against a nematode is provided. The method comprises the step of administering a composition comprising a bacterial strain modified for secretion of a plant defense elicitor peptide to the plant, wherein the bacterial strain comprises an exogenous sequence encoding the plant defense elicitor peptide, and wherein administration of the composition to the plant stimulates defense of the plant against the nematode. The previously described embodiments of the bacterial strain modified for secretion of a plant defense elicitor peptide and the method of controlling root galling in a plant are applicable to the method of stimulating defense of a plant against a nematode described herein.

In an embodiment, stimulating defense against the nematode is associated with control of root galling in the plant. In an embodiment, control of root galling comprises a reduction in root galling disease in the plant. In an embodiment, control of root galling comprises a reduction in root galling disease phenotype in the plant. In an embodiment, the reduction is a reduction of about 20%. In an embodiment, the reduction is a reduction of about 25%. In an embodiment, the reduction is a reduction of about 30%. In an embodiment, the reduction is a reduction of about 40%. In an embodiment, the reduction is a reduction of about 50%.

In an embodiment, control of root galling comprises a reduction in gall number in one or more roots of the plant. In an embodiment, the reduction in gall number is a reduction in average number of galls in a population of plants. In an embodiment, the reduction is a reduction of about 20%. In an embodiment, the reduction is a reduction of about 25%. In an embodiment, the reduction is a reduction of about 30%. In an embodiment, the reduction is a reduction of about 40%. In an embodiment, the reduction is a reduction of about 50%.

The following numbered embodiments are contemplated and are non-limiting:
1. A bacterial strain modified for secretion of a plant defense elicitor peptide, said bacterial strain comprising an exogenous sequence encoding the plant defense elicitor peptide.
2. The bacterial strain of clause 1, any other suitable clause, or any combination of suitable clauses, wherein the bacterial strain is a Gram-positive bacterial strain.
3. The bacterial strain of clause 1, any other suitable clause, or any combination of suitable clauses, wherein the bacterial strain is a Gram-negative bacterial strain.
4. The bacterial strain of clause 1, any other suitable clause, or any combination of suitable clauses, wherein the bacterial strain is a Bacillus bacterial strain.
5. The bacterial strain of clause 4, any other suitable clause, or any combination of suitable clauses, wherein the Bacillus bacterial strain is a root-associating Bacillus strain.
6. The bacterial strain of clause 4, any other suitable clause, or any combination of suitable clauses, wherein the Bacillus bacterial strain is Bacillus subtilis.
7. The bacterial strain of clause 1, any other suitable clause, or any combination of suitable clauses, wherein the bacterial strain is an Escherichia bacterial strain.
8. The bacterial strain of clause 7, any other suitable clause, or any combination of suitable clauses, wherein the Escherichia bacterial strain is a root-associating Escherichia strain.
9. The bacterial strain of clause 7, any other suitable clause, or any combination of suitable clauses, wherein the Escherichia bacterial strain is Escherichia coli.
10. The bacterial strain of clause 1, any other suitable clause, or any combination of suitable clauses, wherein the plant defense elicitor peptide is StPep1.
11. The bacterial strain of clause 1, any other suitable clause, or any combination of suitable clauses, wherein the plant defense elicitor peptide is SlPep6.
12. The bacterial strain of clause 1, any other suitable clause, or any combination of suitable clauses, wherein the plant defense elicitor peptide is AtPep1.
13. The bacterial strain of clause 1, any other suitable clause, or any combination of suitable clauses, wherein the exogenous sequence encoding the plant defense elicitor peptide is codon optimized.
14. The bacterial strain of clause 1, any other suitable clause, or any combination of suitable clauses, wherein the exogenous sequence encoding the plant defense elicitor peptide is SEQ ID NO: 1 (ATERRGRPPSRPKVGSGPPPQNN).
15. The bacterial strain of clause 1, any other suitable clause, or any combination of suitable clauses, wherein the exogenous sequence encoding the plant defense elicitor peptide is a sequence having at least 95% sequence identity to SEQ ID NO: 1.
16. The bacterial strain of clause 1, any other suitable clause, or any combination of suitable clauses, wherein the exogenous sequence encoding the plant defense elicitor peptide is SEQ ID NO: 2 (ATDRRGRPPSRPKVGSGPPPQNN).
17. The bacterial strain of clause 1, any other suitable clause, or any combination of suitable clauses, wherein the exogenous sequence encoding the plant defense elicitor peptide is a sequence having at least 95% sequence identity to SEQ ID NO: 2.
18. The bacterial strain of clause 1, any other suitable clause, or any combination of suitable clauses, wherein the exogenous sequence encoding the plant defense elicitor peptide is SEQ ID NO: 3: ATKVKAKQRGKEKVSSGRPGQHN.
19. The bacterial strain of clause 1, any other suitable clause, or any combination of suitable clauses, wherein the exogenous sequence encoding the plant defense elicitor peptide is a sequence having at least 95% sequence identity to SEQ ID NO: 3.
20. The bacterial strain of clause 1, any other suitable clause, or any combination of suitable clauses, wherein the strain is the Bacillus subtilis strain deposited with Agriculture Research Culture Collection (NRRL) selected from the group consisting of Bacillus subtilis RIK1285; BsStPep1; assigned an accession number B-68058, Bacillus subtilis RIK1285; BsSlPep6; assigned an accession number B-68059, and Bacillus subtilis RIK1285; BsAtPep1; assigned an accession number B-68057.
21. The bacterial strain of clause 1, any other suitable clause, or any combination of suitable clauses, wherein the modified strain affects the expression of at least 5 marker genes for the plant defense elicitor peptide.
22. The bacterial strain of clause 21, any other suitable clause, or any combination of suitable clauses, wherein the marker genes for the plant defense elicitor peptide are found in a plant root.
23. The bacterial strain of clause 22, any other suitable clause, or any combination of suitable clauses, wherein the plant root is a monocot plant root.
24. The bacterial strain of clause 22, any other suitable clause, or any combination of suitable clauses, wherein the plant root is a dicot plant root.
25. The bacterial strain of clause 22, any other suitable clause, or any combination of suitable clauses, wherein the plant root is a potato plant root.
26. The bacterial strain of clause 22, any other suitable clause, or any combination of suitable clauses, wherein the plant root is a tomato plant root.
27. The bacterial strain of clause 22, any other suitable clause, or any combination of suitable clauses, wherein the plant root is an Arabidopsis plant root.
28. The bacterial strain of clause 1, any other suitable clause, or any combination of suitable clauses, wherein the modified strain amplifies innate immunity of a plant.
29. The bacterial strain of clause 28, any other suitable clause, or any combination of suitable clauses, wherein the plant is a monocot plant.
30. The bacterial strain of clause 28, any other suitable clause, or any combination of suitable clauses, wherein the plant is a dicot plant.
31. The bacterial strain of clause 28, any other suitable clause, or any combination of suitable clauses, wherein the plant is a potato plant.
32. The bacterial strain of clause 28, any other suitable clause, or any combination of suitable clauses, wherein the plant is a tomato plant.
33. The bacterial strain of clause 28, any other suitable clause, or any combination of suitable clauses, wherein the plant is an Arabidopsis plant.
34. The bacterial strain of clause 1, any other suitable clause, or any combination of suitable clauses, wherein the modified strain is configured to colonize one or more roots of a plant for at least 7 days.
35. The bacterial strain of clause 1, any other suitable clause, or any combination of suitable clauses, wherein the modified strain is configured to colonize one or more roots of a plant for at least 10 days.
36. The bacterial strain of clause 1, any other suitable clause, or any combination of suitable clauses, wherein the modified strain is configured to colonize one or more roots of a plant for at least 12 days.
37. The bacterial strain of clause 1, any other suitable clause, or any combination of suitable clauses, wherein the modified strain is configured to colonize one or more roots of a plant for at least 14 days.
38. The bacterial strain of clause 1, any other suitable clause, or any combination of suitable clauses, wherein the modified strain is configured to colonize one or more roots of a plant for at least 21 days.
39. The bacterial strain of clause 1, any other suitable clause, or any combination of suitable clauses, wherein the modified strain is configured to colonize one or more roots of a plant for at least 28 days.
40. The bacterial strain of clause 1, any other suitable clause, or any combination of suitable clauses, wherein the modified strain is configured to colonize one or more roots of a plant for at least 30 days.
41. The bacterial strain of clause 40, any other suitable clause, or any combination of suitable clauses, wherein the plant is a monocot plant.
42. The bacterial strain of clause 40, any other suitable clause, or any combination of suitable clauses, wherein the plant is a dicot plant.
43. The bacterial strain of clause 40, any other suitable clause, or any combination of suitable clauses, wherein the plant is a potato plant.
44. The bacterial strain of clause 40, any other suitable clause, or any combination of suitable clauses, wherein the plant is a tomato plant.
45. The bacterial strain of clause 40, any other suitable clause, or any combination of suitable clauses, wherein the plant is an Arabidopsis plant.
46. The bacterial strain of clause 1, wherein the modified strain is configured to colonize one or more roots of a plant and increase resistance of the plant to a root-knot nematode.
47. The bacterial strain of clause 46, any other suitable clause, or any combination of suitable clauses, wherein the plant is a monocot plant.
48. The bacterial strain of clause 46, any other suitable clause, or any combination of suitable clauses, wherein the plant is a dicot plant.
49. The bacterial strain of clause 46, any other suitable clause, or any combination of suitable clauses, wherein the plant is a potato plant.
50. The bacterial strain of clause 46, any other suitable clause, or any combination of suitable clauses, wherein the plant is a tomato plant.
51. The bacterial strain of clause 46, any other suitable clause, or any combination of suitable clauses, wherein the plant is an Arabidopsis plant.
52. The bacterial strain of clause 46, any other suitable clause, or any combination of suitable clauses, wherein the root-knot nematode is a Meloidogyne nematode.
53. The bacterial strain of clause 52, any other suitable clause, or any combination of suitable clauses, wherein the Meloidogyne nematode is Meloidogyne chitwoodi, Meloidogyne incognita, or both.
54. The bacterial strain of clause 52, any other suitable clause, or any combination of suitable clauses, wherein the Meloidogyne nematode is Meloidogyne chitwoodi.
55. The bacterial strain of clause 52, any other suitable clause, or any combination of suitable clauses, wherein the Meloidogyne nematode is Meloidogyne incognita.
56. An E. coli or B. subtilis bacterial expression vector comprising:
   a nucleic acid sequence encoding a plant defense elicitor peptide;
   a bacterial promoter functional in E. coli and/or B. subtilis, wherein said promoter is operably linked to said defense elicitor peptide encoding sequence;
   a nucleic acid sequence encoding a secretory signal peptide, wherein said secretory signal peptide encoding sequence is operably linked to said defense elicitor peptide encoding sequence; and
   a sequence encoding a selectable marker.
57. The bacterial expression vector of clause 56, any other suitable clause, or any combination of suitable clauses, wherein the plant defense elicitor peptide is selected from the group consisting of StPep1 (SEQ ID NO: 1: ATERRGRPPSRPKVGSGPPPQNN), SlPep6 (SEQ ID NO: 2: ATDRRGRPPSRPKVGSGPPPQNN) and AtPep1 (SEQ ID NO: 3: ATKVKAKQRGKEKVSSGRPGQHN).
58. The bacterial expression vector of clause 56, any other suitable clause, or any combination of suitable clauses, wherein the promoter is aprE and the secretory signal peptide is aprE SP.
59. The bacterial expression vector of clause 56, any other suitable clause, or any combination of suitable clauses, wherein the bacterial expression vector further comprises 1 to 16 UAS sequences operably linked to said promoter sequence.
60. The bacterial expression vector of clause 56, any other suitable clause, or any combination of suitable clauses, wherein said selectable marker is an antibiotic resistance gene product.
61. The bacterial expression vector of clause 56, any other suitable clause, or any combination of suitable clauses, wherein the bacterial expression vector further comprises a replication region for E. coli.
62. The bacterial expression vector of clause 56, any other suitable clause, or any combination of suitable clauses, wherein the bacterial expression vector further comprises a replication region for B. subtilis.
63. A method of controlling root galling in a plant, said method comprising the step of administering a composition comprising a bacterial strain modified for secretion of a plant defense elicitor peptide to the plant,
   wherein the bacterial strain comprises an exogenous sequence encoding the plant defense elicitor peptide, and
   wherein administration of the composition to the plant controls root galling in the plant.
64. The method of clause 63, any other suitable clause, or any combination of suitable clauses, wherein controlling root galling comprises a reduction in root galling disease in the plant.
65. The method of clause 63, any other suitable clause, or any combination of suitable clauses, wherein controlling root galling comprises a reduction in root galling disease phenotype in the plant.
66. The method of clause 65, any other suitable clause, or any combination of suitable clauses, wherein the reduction is a reduction of about 20%.
67. The method of clause 65, any other suitable clause, or any combination of suitable clauses, wherein the reduction is a reduction of about 25%.
68. The method of clause 65, any other suitable clause, or any combination of suitable clauses, wherein the reduction is a reduction of about 30%.
69. The method of clause 65, any other suitable clause, or any combination of suitable clauses, wherein the reduction is a reduction of about 40%.
70. The method of clause 65, any other suitable clause, or any combination of suitable clauses, wherein the reduction is a reduction of about 50%.
71. The method of clause 63, any other suitable clause, or any combination of suitable clauses, wherein controlling root galling comprises a reduction in gall number in one or more roots of the plant.
72. The method of clause 71, any other suitable clause, or any combination of suitable clauses, wherein the reduction in gall number is a reduction in average number of galls in a population of plants.
73. The method of clause 72, any other suitable clause, or any combination of suitable clauses, wherein the reduction is a reduction of about 20%.
74. The method of clause 72, any other suitable clause, or any combination of suitable clauses, wherein the reduction is a reduction of about 25%.
75. The method of clause 72, any other suitable clause, or any combination of suitable clauses, wherein the reduction is a reduction of about 30%.
76. The method of clause 72, any other suitable clause, or any combination of suitable clauses, wherein the reduction is a reduction of about 40%.
77. The method of clause 72, any other suitable clause, or any combination of suitable clauses, wherein the reduction is a reduction of about 50%.
78. The method of clause 63, any other suitable clause, or any combination of suitable clauses, wherein the plant is a monocot plant.
79. The method of clause 63, any other suitable clause, or any combination of suitable clauses, wherein the plant is a dicot plant.
80. The method of clause 63, any other suitable clause, or any combination of suitable clauses, wherein the plant is a potato plant.
81. The method of clause 63, any other suitable clause, or any combination of suitable clauses, wherein the plant is a tomato plant.
82. The method of clause 63, any other suitable clause, or any combination of suitable clauses, wherein the plant is an Arabidopsis plant.
83. The method of clause 63, any other suitable clause, or any combination of suitable clauses, wherein the composition comprises a liquid.
84. The method of clause 83, any other suitable clause, or any combination of suitable clauses, wherein the liquid is water.
85. The method of clause 63, any other suitable clause, or any combination of suitable clauses, wherein the administration is applied via irrigation.
86. The method of clause 63, any other suitable clause, or any combination of suitable clauses, wherein the bacterial strain is a Gram-positive bacterial strain.
87. The method of clause 63, any other suitable clause, or any combination of suitable clauses, wherein the bacterial strain is a Gram-negative bacterial strain.
88. The method of clause 63, any other suitable clause, or any combination of suitable clauses, wherein the bacterial strain is a Bacillus bacterial strain.
89. The method of clause 88, any other suitable clause, or any combination of suitable clauses, wherein the Bacillus bacterial strain is a root-associating Bacillus strain.
90. The method of clause 88, any other suitable clause, or any combination of suitable clauses, wherein the Bacillus bacterial strain is Bacillus subtilis.
91. The method of clause 63, any other suitable clause, or any combination of suitable clauses, wherein the bacterial strain is an Escherichia bacterial strain.
92. The method of clause 91, any other suitable clause, or any combination of suitable clauses, wherein the Escherichia bacterial strain is a root-associating Escherichia strain.
93. The method of clause 91, any other suitable clause, or any combination of suitable clauses, wherein the Escherichia bacterial strain is Escherichia coli.
94. The method of clause 63, any other suitable clause, or any combination of suitable clauses, wherein the plant defense elicitor peptide is StPep1.
95. The method of clause 63, any other suitable clause, or any combination of suitable clauses, wherein the plant defense elicitor peptide is SlPep6.
96. The method of clause 63, any other suitable clause, or any combination of suitable clauses, wherein the plant defense elicitor peptide is AtPep1.
97. The method of clause 63, any other suitable clause, or any combination of suitable clauses, wherein the exogenous sequence encoding the plant defense elicitor peptide is codon optimized.
98. The method of clause 63, any other suitable clause, or any combination of suitable clauses, wherein the exogenous sequence encoding the plant defense elicitor peptide is

SEQ ID NO: 1
(ATERRGRPPSRPKVGSGPPPQNN).

99. The method of clause 63, any other suitable clause, or any combination of suitable clauses, wherein the exogenous sequence encoding the plant defense elicitor peptide is a sequence having at least 95% sequence identity to SEQ ID NO: 1.
100. The method of clause 63, any other suitable clause, or any combination of suitable clauses, wherein the exogenous sequence encoding the plant defense elicitor peptide is

SEQ ID NO: 2
(ATDRRGRPPSRPKVGSGPPPQNN).

101. The method of clause 63, any other suitable clause, or any combination of suitable clauses, wherein the exogenous sequence encoding the plant defense elicitor peptide is a sequence having at least 95% sequence identity to SEQ ID NO: 2.
102. The method of clause 63, any other suitable clause, or any combination of suitable clauses, wherein the exogenous sequence encoding the plant defense elicitor peptide is

SEQ ID NO: 3:
ATKVKAKQRGKEKVSSGRPGQHN.

103. The method of clause 63, any other suitable clause, or any combination of suitable clauses, wherein the exogenous sequence encoding the plant defense elicitor peptide is a sequence having at least 95% sequence identity to SEQ ID NO: 3.
104. The method of clause 63, any other suitable clause, or any combination of suitable clauses, wherein the strain is the Bacillus subtilis strain deposited with Agriculture Research Culture Collection (NRRL) selected from the group consisting of Bacillus subtilis RIK1285; BsStPep1; assigned an accession number B-68058, Bacillus subtilis RIK1285; BsSlPep6; assigned an accession number B-68059, and Bacillus subtilis RIK1285; BsAtPep1; assigned an accession number B-68057.
105. The method of clause 63, any other suitable clause, or any combination of suitable clauses, wherein the root galling is caused by a root-knot nematode.
106. The method of clause 105, any other suitable clause, or any combination of suitable clauses, wherein the root-knot nematode is a Meloidogyne nematode.
107. The method of clause 105, any other suitable clause, or any combination of suitable clauses, wherein the Meloidogyne nematode is Meloidogyne chitwoodi, Meloidogyne incognita, or both.
108. The method of clause 105, any other suitable clause, or any combination of suitable clauses, wherein the Meloidogyne nematode is Meloidogyne chitwoodi.
109. The method of clause 105, any other suitable clause, or any combination of suitable clauses, wherein the Meloidogyne nematode is Meloidogyne incognita.
110. The method of clause 63, any other suitable clause, or any combination of suitable clauses, wherein administering the composition is performed one time per week.
111. The method of clause 63, any other suitable clause, or any combination of suitable clauses, wherein administering the composition is performed one time every 10 days.
112. The method of clause 63, any other suitable clause, or any combination of suitable clauses, wherein administering the composition is performed one time every 14 days.
113. The method of clause 63, any other suitable clause, or any combination of suitable clauses, wherein administering the composition to the plant is performed by treating the soil near or adjacent to the plant.
114. The method of clause 63, any other suitable clause, or any combination of suitable clauses, wherein administering the composition to the plant is performed by treating the soil near or adjacent to one or more roots of the plant.
115. The method of clause 63, any other suitable clause, or any combination of suitable clauses, wherein administering the composition to the plant is performed by treating the plant upon emergence of the plant.
116. The method of clause 63, any other suitable clause, or any combination of suitable clauses, wherein administering the composition to the plant is performed by treating the plant prior to emergence of the plant.
117. A method of stimulating defense of a plant against a nematode, said method comprising the step of administering a composition comprising a bacterial strain modified for secretion of a plant defense elicitor peptide to the plant,
   wherein the bacterial strain comprises an exogenous sequence encoding the plant defense elicitor peptide, and
   wherein administration of the composition to the plant stimulates defense of the plant against the nematode.
118. The method of clause 117, any other suitable clause, or any combination of suitable clauses, wherein stimulating defense against the nematode is associated with control of root galling in the plant.
119. The method of clause 118, any other suitable clause, or any combination of suitable clauses, wherein control of root galling comprises a reduction in root galling disease in the plant.
120. The method of clause 118, any other suitable clause, or any combination of suitable clauses, wherein control of root galling comprises a reduction in root galling disease phenotype in the plant.
121. The method of clause 120, any other suitable clause, or any combination of suitable clauses, wherein the reduction is a reduction of about 20%.
122. The method of clause 120, any other suitable clause, or any combination of suitable clauses, wherein the reduction is a reduction of about 25%.
123. The method of clause 120, any other suitable clause, or any combination of suitable clauses, wherein the reduction is a reduction of about 30%.
124. The method of clause 120, any other suitable clause, or any combination of suitable clauses, wherein the reduction is a reduction of about 40%.
125. The method of clause 120, any other suitable clause, or any combination of suitable clauses, wherein the reduction is a reduction of about 50%.
126. The method of clause 118, any other suitable clause, or any combination of suitable clauses, wherein control of root galling comprises a reduction in gall number in one or more roots of the plant.
127. The method of clause 126, any other suitable clause, or any combination of suitable clauses, wherein the reduction in gall number is a reduction in average number of galls in a population of plants.
128. The method of clause 127, any other suitable clause, or any combination of suitable clauses, wherein the reduction is a reduction of about 20%.
129. The method of clause 127, any other suitable clause, or any combination of suitable clauses, wherein the reduction is a reduction of about 25%.
130. The method of clause 127, any other suitable clause, or any combination of suitable clauses, wherein the reduction is a reduction of about 30%.
131. The method of clause 127, any other suitable clause, or any combination of suitable clauses, wherein the reduction is a reduction of about 40%.
132. The method of clause 127, any other suitable clause, or any combination of suitable clauses, wherein the reduction is a reduction of about 50%.
133. The method of clause 117, any other suitable clause, or any combination of suitable clauses, wherein the plant is a monocot plant.
134. The method of clause 117, any other suitable clause, or any combination of suitable clauses, wherein the plant is a dicot plant.
135. The method of clause 117, any other suitable clause, or any combination of suitable clauses, wherein the plant is a potato plant.
136. The method of clause 117, any other suitable clause, or any combination of suitable clauses, wherein the plant is a tomato plant.
137. The method of clause 117, any other suitable clause, or any combination of suitable clauses, wherein the plant is an Arabidopsis plant.
138. The method of clause 117, any other suitable clause, or any combination of suitable clauses, wherein the composition comprises a liquid.
139. The method of clause 138, any other suitable clause, or any combination of suitable clauses, wherein the liquid is water.
140. The method of clause 117, any other suitable clause, or any combination of suitable clauses, wherein the administration is applied via irrigation.
141. The method of clause 117, any other suitable clause, or any combination of suitable clauses, wherein the bacterial strain is a Gram-positive bacterial strain.
142. The method of clause 117, any other suitable clause, or any combination of suitable clauses, wherein the bacterial strain is a Gram-negative bacterial strain.
143. The method of clause 117, any other suitable clause, or any combination of suitable clauses, wherein the bacterial strain is a Bacillus bacterial strain.
144. The method of clause 143, any other suitable clause, or any combination of suitable clauses, wherein the Bacillus bacterial strain is a root-associating Bacillus strain.
145. The method of clause 143, any other suitable clause, or any combination of suitable clauses, wherein the Bacillus bacterial strain is Bacillus subtilis.
146. The method of clause 117, any other suitable clause, or any combination of suitable clauses, wherein the bacterial strain is an Escherichia bacterial strain.
147. The method of clause 146, any other suitable clause, or any combination of suitable clauses, wherein the Escherichia bacterial strain is a root-associating Escherichia strain.
148. The method of clause 146, any other suitable clause, or any combination of suitable clauses, wherein the Escherichia bacterial strain is Escherichia coli.
149. The method of clause 117, any other suitable clause, or any combination of suitable clauses, wherein the plant defense elicitor peptide is StPep1.
150. The method of clause 117, any other suitable clause, or any combination of suitable clauses, wherein the plant defense elicitor peptide is SlPep6.
151. The method of clause 117, any other suitable clause, or any combination of suitable clauses, wherein the plant defense elicitor peptide is AtPep1.
152. The method of clause 117, any other suitable clause, or any combination of suitable clauses, wherein the exogenous sequence encoding the plant defense elicitor peptide is codon optimized.
153. The method of clause 117, any other suitable clause, or any combination of suitable clauses, wherein the exogenous sequence encoding the plant defense elicitor peptide is

SEQ ID NO: 1
(ATERRGRPPSRPKVGSGPPPQNN).

154. The method of clause 117, any other suitable clause, or any combination of suitable clauses, wherein the exogenous sequence encoding the plant defense elicitor peptide is a sequence having at least 95% sequence identity to SEQ ID NO: 1.
155. The method of clause 117, any other suitable clause, or any combination of suitable clauses, wherein the exogenous sequence encoding the plant defense elicitor peptide is

SEQ ID NO: 2
(ATDRRGRPPSRPKVGSGPPPQNN).

156. The method of clause 117, any other suitable clause, or any combination of suitable clauses, wherein the exogenous sequence encoding the plant defense elicitor peptide is a sequence having at least 95% sequence identity to SEQ ID NO: 2.
157. The method of clause 117, any other suitable clause, or any combination of suitable clauses, wherein the exogenous sequence encoding the plant defense elicitor peptide is

SEQ ID NO: 3:
ATKVKAKQRGKEKVSSGRPGQHN.

158. The method of clause 117, any other suitable clause, or any combination of suitable clauses, wherein the exogenous sequence encoding the plant defense elicitor peptide is a sequence having at least 95% sequence identity to SEQ ID NO: 3.
159. The method of clause 117, any other suitable clause, or any combination of suitable clauses, wherein the strain is the Bacillus subtilis strain deposited with Agriculture Research Culture Collection (NRRL) selected from the group consisting of Bacillus subtilis RIK1285; BsStPep1; assigned an accession number B-68058, Bacillus subtilis RIK1285; BsSlPep6; assigned an accession number B-68059, and Bacillus subtilis RIK1285; BsAtPep1; assigned an accession number B-68057.
160. The method of clause 117, any other suitable clause, or any combination of suitable clauses, wherein the nematode is a root-knot nematode.
161. The method of clause 160, any other suitable clause, or any combination of suitable clauses, wherein the root-knot nematode is a Meloidogyne nematode is a Meloidogyne nematode.
162. The method of clause 161, any other suitable clause, or any combination of suitable clauses, wherein the Meloidogyne nematode is Meloidogyne chitwoodi, Meloidogyne incognita, or both.
163. The method of clause 161, any other suitable clause, or any combination of suitable clauses, wherein the Meloidogyne nematode is Meloidogyne chitwoodi.
164. The method of clause 161, any other suitable clause, or any combination of suitable clauses, wherein the Meloidogyne nematode is Meloidogyne incognita.
165. The method of clause 117, any other suitable clause, or any combination of suitable clauses, wherein administering the composition is performed one time per week.
166. The method of clause 117, any other suitable clause, or any combination of suitable clauses, wherein administering the composition is performed one time every 10 days.
167. The method of clause 117, any other suitable clause, or any combination of suitable clauses, wherein administering the composition is performed one time every 14 days.
168. The method of clause 117, any other suitable clause, or any combination of suitable clauses, wherein administering the composition to the plant is performed by treating the soil near or adjacent to the plant.
169. The method of clause 117, any other suitable clause, or any combination of suitable clauses, wherein administering the composition to the plant is performed by treating the soil near or adjacent to one or more roots of the plant.
170. The method of clause 117, any other suitable clause, or any combination of suitable clauses, wherein administering the composition to the plant is performed by treating the plant upon emergence of the plant.
171. The method of clause 117, any other suitable clause, or any combination of suitable clauses, wherein administering the composition to the plant is performed by treating the plant prior to emergence of the plant.

### EXAMPLES

### Example 1

### Effect of StPep1 Administration on Potatoes

The instant example was performed to determine whether the potato defense elicitor peptide StPep1 is able to protect particular dicot roots (e.g., potato roots) from root-knot nematode pathogenic infection. The exemplary root-knot nematode M. chitwoodi was analyzed in the instant example to evaluate Russet Burbank (RB) potato plants that were pre-treated with StPep1.

M. chitwoodi isolate Roza (McRoza)3 was maintained on the susceptible tomato Solanum lycopersicum cv. Rutgers under greenhouse conditions. Nematode eggs were collected from tomato roots by cutting them into small pieces and agitating them in a 0.6% sodium hypochlorite solution for 3 minutes. The solution was then poured over a set of sieves (pore size of 125, 45, 25 μm). Eggs were collected on the 25 μm sieve and then further purified using sucrose floatation. The eggs were suspended in 0.1% Plant Preservative Mixture (Plant Cell Technology) and incubated in a modified Baermann pan at room temperature in darkness. Hatched infective J2s were collected after 3 days of incubation.

Three-four-week-old Russet Burbank tissue culture plantlets were transferred to 500-mL cones filled with sand and were kept under growth chamber conditions (14-hour light/10-hour darkness, 23° C.) for 14 days. The potato plants were then watered with 10 ml of synthetic 1 μM StPep1 solution. Two days after peptide treatment, 500 freshly hatched J2s of M. chitwoodi were used to inoculate the potato plants. The plants were harvested at 12-day-post-inoculation and the number of galls was recorded. The fresh weights of roots and shoots were also measured.

To count egg masses, plants were harvested at 35-day-post inoculation, and roots were stained with phloxin B solution (0.15 g/L) to visualize egg masses. For nematode invasion assay, plants were harvested at 4-day-post inoculation, and roots were stained with acid fuchsin solution (0.35% acid fuchsin, 25% acetic acid) to visualize parasitic J2s inside potato roots.

To test if StPep1 demonstrated negative effects on the viability of nematode J2, approximately 40 J2s of M. chitwoodi were incubated in 1 μM of StPep1 solution or in mock solution in 24-well plates at room temperature in dark. The numbers of live and dead J2s were counted at 2, 4 and 7 days later under a dissecting microscope. J2s were scored as dead if their bodies were straight in shape and did not move. Percentage of live J2s was calculated. Each treatment included ten biological samples and was repeated twice.

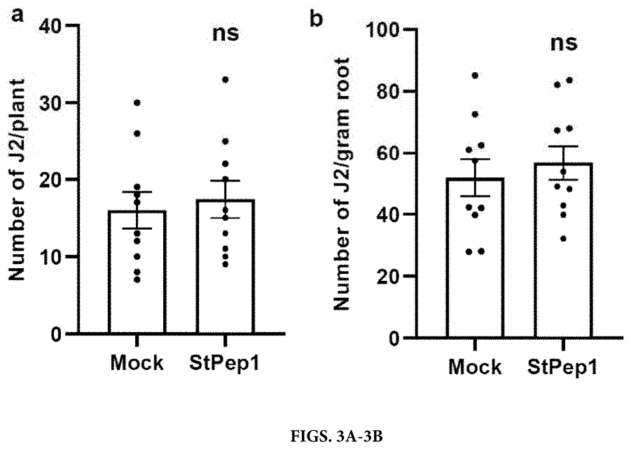

Further, potato plants were watered with either a 1 μM StPep1 or a mock solution two days prior to nematode inoculation. Nematode juveniles were applied to the roots, and four days after M. chitwoodi inoculation, the number of nematodes in the roots were counted. There was no observed difference in the number of juveniles inside the potato roots at 4 days post infection (dpi) between the StPep1 and mock treated and plants, indicating that StPep1 has no effect on nematode penetration of the roots (FIGS. 3A-3B).

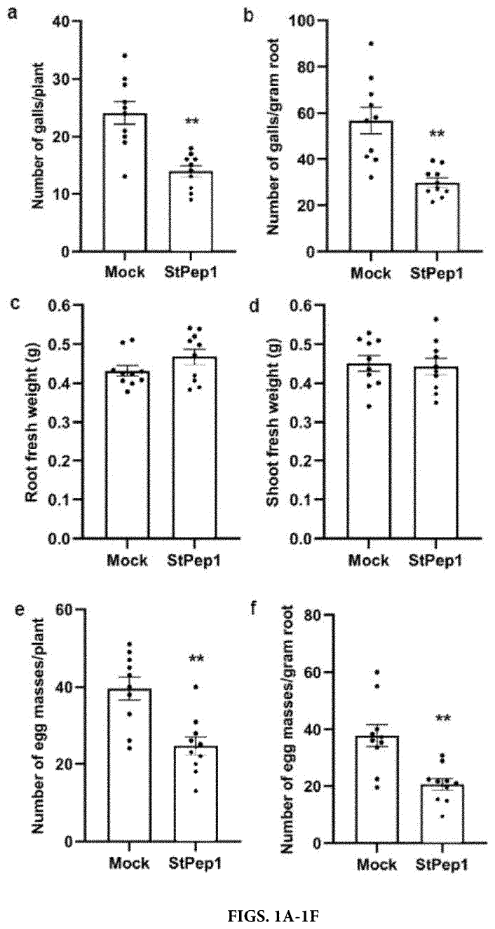

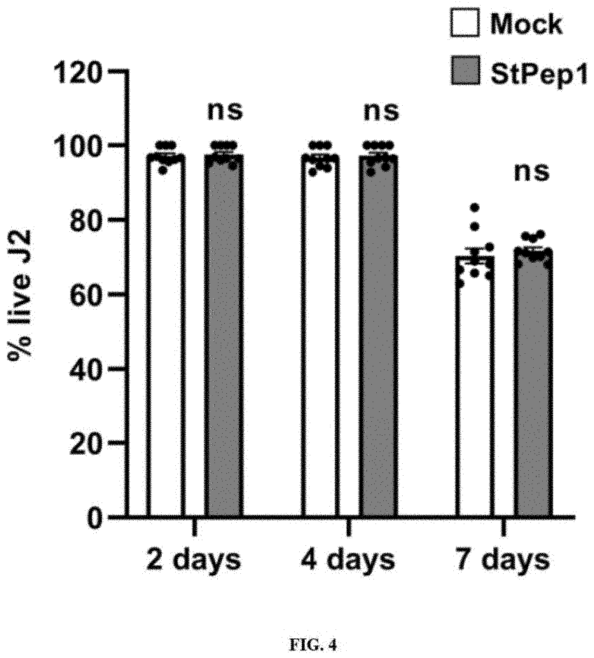

Surprisingly, StPep1 pre-treatment significantly reduced M. chitwoodi galling (FIGS. 1A and 1B) and reduced the number of egg masses (FIGS. 1E and 1F) produced on potato roots compared to the control. Because of the potential trade-off between plant growth and defense, the root and shoot biomasses of infected potato plants were measured and no negative effects were observed on potato above-ground weights or root weights by StPep1 treatment (FIGS. 1C and 1D). The StPep1 solution did not directly kill the nematodes (FIG. 4), suggesting that the decreased galling was due to enhanced plant resistance to the nematodes. Thus, a single watering with an StPep1 solution was effective to stimulate nematode resistance in plants without measurable adverse effects on plant growth.

### Example 2

### Engineering of Bacterial Strain with StPep1 Defense Elicitor Peptide

The instant example evaluated the efficacy of an engineered bacterial strain modified to secrete a plant defense elicitor peptide. For this example, Bacillus subtilis was used as the exemplary bacterial strain and StPep1 was used as the exemplary plant defense elicitor peptide.

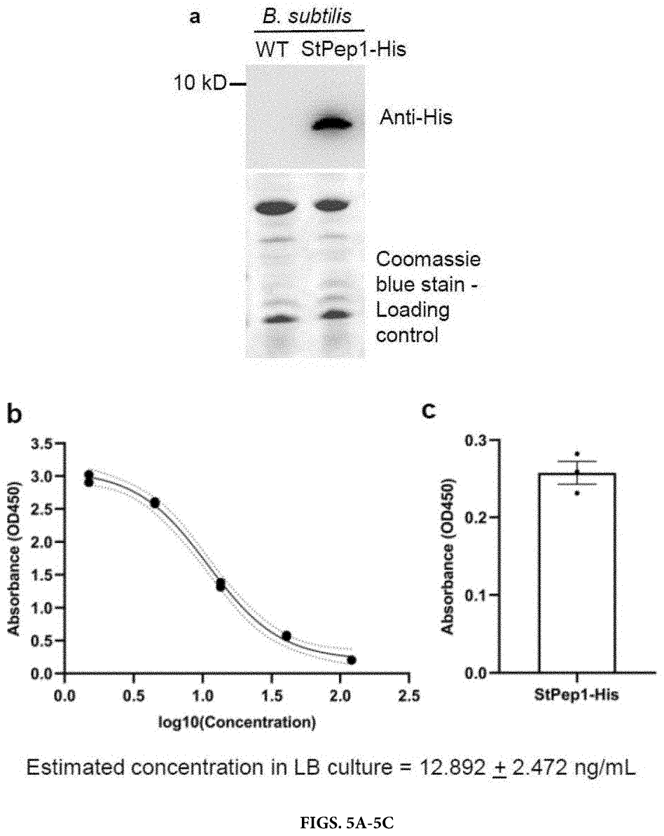

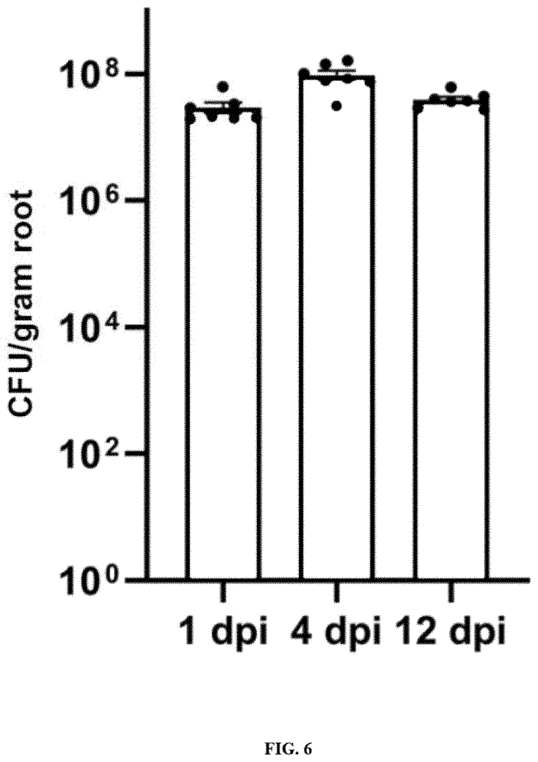

Bacillus subtilis was engineered to secrete His-tagged StPep1 (Bs-StPep1-His). The fusion protein was detected by Western blot in B. subtilis protein extracts (FIG. 5A). Secretion of StPep1 was confirmed by ELISA, which showed approximately 12.89 ng/ml of StPep1 in the culture broth. To determine how long the B. subtilis that secretes StPep1 remains associated with the potato roots during the experiments, the bacterial colony forming units associated with potato roots at 1, 2, and 12 days after inoculation were quantified. The results showed that the bacteria remained in association with the roots at least 12 days post-inoculation (FIG. 6).

The DNA sequence encoding StPep1 was optimized for better expression in the bacterium B. subtilis using the codon optimization tool of IDT (Integrated DNA Technologies, USA). The codon-optimized StPep1 gene was synthesized by PCR using primers with overlapping region, and two versions of the gene were obtained, one with stop codon and the other without stop codon. The primers included:

StPep1-FP:
PCR to clone codon-optimized StPep1 to pBE-S;
SEQ ID NO: 4
(CACCgagctcGCAACCGAGAGAAGAGGTAGACCTCctagccgtcctaaa
gtcggc)
StPep1-RP-NOSTOP:
PCR to clone codon-optimized StPep1 to pBE-S;
SEQ ID NO: 5:
(AGCTtctagaATTGTTCTGCGGCGGAGGCCCGGAgccgactttaggacg
gctag)
StPep1-RP-STOP:
PCR to clone codon-optimzed StPep1 to pBE-S;
SEQ ID NO: 6:
(AGCTtctagaTTAattgttctgcggcggaggc)

For instance, the codon optimized sequence for AtPep1 is identified as

SEQ ID NO: 7
(ACAAAAGTCAAGGCGAAACAACGGGGAAAAGAAAAAGTGTCCAGTGGGA
GACCGGGACAACATAACTGATCTCGGATGCAGTCTACAATGCAG).
The codon optimized sequence for SlPep6is
identified as
SEQ ID NO: 8
(Gcaaccgacagaagaggtcgtcctccatcacgtccaaaggtggggagcg
gccctccaccacaaaataattctagaaagatg).
The codon optimized sequence for StPep6is
identified as
SEQ ID NO: 9
(Gcaaccgagagaagaggtagacctcctagccgtcctaaagtcggcgggc
ctccgccgcagaacaat).

The PCR products were cloned into the pBE-S vector (Takara, USA) via SacI/XbaI enzyme sites. The StPep1 gene without stop codon was fused with the 6*His tag at C-terminus. The ligated plasmids were introduced into E. coli One Shot TOP10 chemically competent cells (Thermo Fisher Scientific, USA). The sequences of the plasmids were confirmed by DNA sequencing (Elim Biopharmaceuticals, USA). The confirmed plasmids were introduced into the B. subtilis RIK1285 by electroporation method. Colony-PCR confirmed the transformation of B. subtilis RIK1285 with the correct plasmids.

Colonies of StPep1-His transformed or wild-type B. subtilis were grown in LB with kanamycin (10m/mL) or LB medium, respectively, at 29° C. overnight. The B. subtilis cultures were diluted in 30-mL of LB medium and kept shaking until OD600=0.8. One mL of the bacterial culture was used for crude protein extraction and expression pression of the StPep1-His fusion protein was detected by western blot using anti-His antibody (Santa Cruz Biotechnology, USA). The supernatants were collected by spinning the rest of the cell cultures. The supernatants were used for purification of His-tagged proteins by the Capturem™ His-Tagged Purification Maxiprep Kit (TaKaRa, USA) according to the manufacturer's instruction. Concentration of His-tagged peptides was estimated using the His Tag Detection Kit (GenScript, USA), a competitive Enzyme-linked immunosorbent assay (ELISA), following the manufacturer's protocol, with horse radish peroxidase reading outputs obtained using the microplate reader SpectraMax Plus384 (Molecular Devices, USA).

### Example 3

### Effect of StPep1 Administration Via Engineered Bacterial Strain on Potatoes

In the instant example, the exemplary StPep1-secreting B. subtilis engineered bacterial strain was assessed to evaluate the improvement in resistance of potato plants against the exemplary root-knot nematode M. chitwoodi.

For Bacillus subtilis pre-treatment, B. subtilis strains were grown in LB medium with kanamycin (10 m/mL) with shaking at 29° C. overnight. The cultures were then diluted by 1:100 in LB medium with kanamycin (10 μg/mL) and incubated with shaking until the OD600 reached 0.6.

The bacterial cultures were spun down and pellets were suspended in one volume deionized H2O. Potato tissue culture plants were immersed with roots in suspensions of B. subtilis strains for two hours before the plants were transferred to sand cone-tainers. Approximately 10 ml of the of B. subtilis suspensions (OD600 0.6) were used to treat the Russet Burbank plants in cones with sand 10 days after transfer. Four days post treatment, the potato plants were inoculated with 500 M. chitwoodi (McRoza) J2s as described above.

Each nematode infection assay for galling or egg masses described above had 10 potato plants and was repeated three times. The nematode invasion assay was repeated twice.

To monitor B. subtilis strain secreting StPep1-His on potato roots, the bacteria were harvested from roots of potato plants at 2, 4, and 12 days post Bacillus inoculation. The potato root system with adhering sand was placed in a tube with 20-mL of sterile water. The tube was vortexed for 1 minute and then treated for 1 minute in an ultrasonic cleaner. A serial dilution of bacterial isolations (10 times from 10-1 to 10-6) were made, and 20 μl of each dilution were dotted on LB selection plates with cycloheximide and kanamycin (100 μg/mL and 10m/mL, respectively). Numbers of colonies were counted ˜20 hours later. CFU per gram root was calculated. The experiment was repeated twice with similar results.

Potato plants were subjected to pre-treatments with water, a B. subtilis culture transformed with the empty B. subtilis expression vector pBE-S, or B. subtilis cultures engineered to express either the C terminus, His-tagged or untagged StPep1. The number of galls per plant at 12 days post-nematode inoculation was counted.

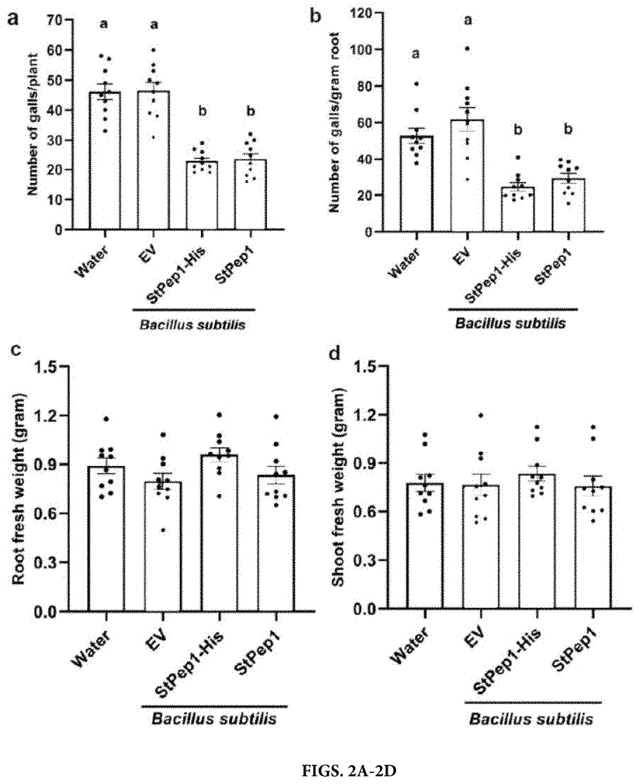

Galling was observed to be significantly reduced on the potatoes pre-treated with the B. subtilis cultures secreting StPep-His1 or untagged StPep1 (FIGS. 2A and 2B). There was no significant difference in galling between pre-treatments of water and the empty vector-transformed B. subtilis (FIGS. 2A and 2B), indicating that this strain of B. subtilis does not induce resistance on its own or have nematocidal activity. Infected plant root and shoot biomasses were not affected by any of the B. subtilis treatments (FIGS. 2C and 2D).

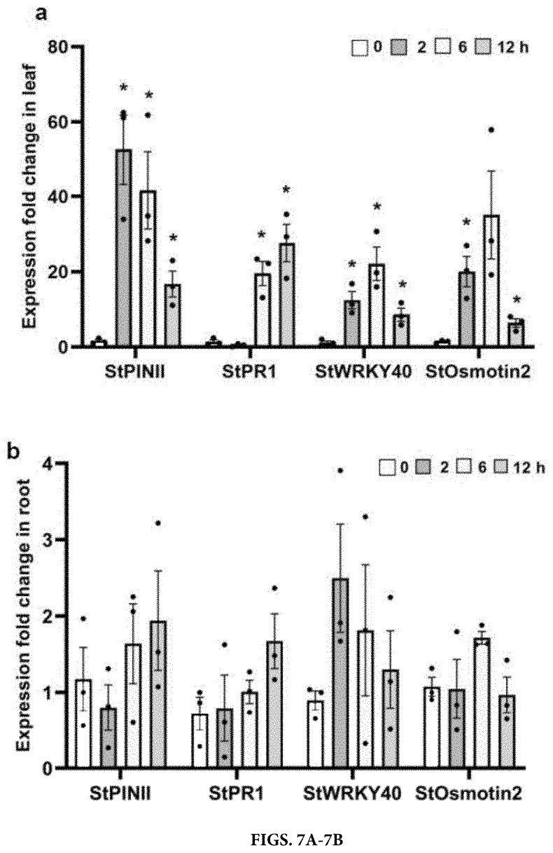

The results show that pre-treatments with engineered B. subtilis expressing StPep1-His or StPep1 can effectively improve resistance of potato plants against M. chitwoodi. To investigate the StPep1 mode of action in potatoes, potato roots were treated with either water (mock) or a 100 nM StPep1 solution for gene expression analyses. A lower concentration of StPep1 solution was chosen because, for these experiments, tissue-cultured potato roots in the StPep1 solution were directly soaked. This is in contrast to previous experiments with StPep1, where watered plants were grown in sand cone-tainers with an StPep1 solution one time prior to nematode infections and subsequent watering. Potato gene expression was measured by quantitative real time PCR (qRT-PCR) at 0, 2, 6, and 12 hour-post-treatment (HPT). Because A. thaliana PEPR signaling is mediated through salicylic acid, jasmonic acid, and ethylene pathways, the expression of four potato genes linked to these signaling pathways were tested in potatoes. The fold change in gene expression were compared between water and StPep1 treatments in roots and leaves in this time course experiment. In potato leaves, StPINII, StOsmotin2, StPR1, and StWRKY40 showed increased expression during the time course study, suggesting a systemic response to the StPep1 root treatment (FIG. 7A). However, none of these genes were differentially regulated in roots (FIG. 7B).

### Example 4

### Identification of Genes Differentially Regulated by StPep1 in Potato Roots

To find genes differentially regulated by StPep1 in potato roots, a transcriptome analysis was performed on Russet Burbank potato roots incubated in a 100 nM StPep1 solution for 0 and 6 hours.

Three-week-old Russet Burbank potato tissue culture plants were treated with a StPep1 solution (100 nM) for 0 and 6 hours by root soaking. Roots from two individual plants were combined as one biological replicate, and three biological replicates were collected for each treatment. Total RNA was extracted from root samples using RNeasy Plant Mini Kit (QIAGEN, Germany). High quality total RNA (3 μg) was sent to Novogene Co. Ltd for library construction and sequencing by the Illumina platform PE150.

Potato tissue culture plants were incubated by immersing roots in a 6-well plate with deionized H₂O for 24 hours with gentle shaking at 80 rpm. Then concentrated bacterial suspensions in water of B. subtilis transformed with pBE-S empty vector or StPep1-His were added to wells of 6-well plates to reach an OD600 of 0.8-1.0. The plates were shaken at 80 rpm. Root samples were harvested at 6 hours of treatment for total RNA extraction and qRT-PCR as described above.

For gene expression analysis, 3-week-old potato tissue culture plants were first incubated by immersing the roots in deionized H₂O for 24 hours. The water was replaced by 100 nM of StPep1 solution or water. The StPep1 (amino acid sequence: ATERRGRPPSRPKVGSGPPPQNN) was synthesized by GenScript, USA. Root or leaf samples were harvested at indicated treatment times. The Russet Burbank potato cultivar was used in all experiments, unless stated otherwise.

The StCOI1-RNAi potato and its corresponding wild-type lines were in the Desiree background. The NahG-expressing potato line and its corresponding wild-type are in the Bannock Russet background. Total RNA was extracted from frozen leaf or root samples using RNeasy Plant Mini Kit (QIAGEN, Germany) according to the manufacturer's instructions. cDNA was produced using ProtoScript II First Strand cDNA Synthesis Kit (New England Biolabs, USA) with oligo-dT primer. qPCR was performed using SsoAdvanced™ Universal SYBR® Green Supermix on a CFX96 Real-Time PCR Detection System (Bio-Rad, USA). The qPCR conditions were: 95° C. for 3 minutes, 40 cycles of 95° C. for 15 seconds, 53° C. for 15 seconds, and 72° C. for 20 seconds, and followed by a melting curve analysis from 65° C. to 95° C. with 0.5° C. increased incrementally. Expression levels of genes of interest in potato were normalized to the expression of potato housekeeping gene elongation factor 1-alpha (StEF1 α). The relative expression levels of each target gene by StPep1 treatment were calculated by comparing with those of corresponding mock treatments using the 2-ΔΔCt method.

The raw sequencing data were trimmed using Trimmomatic-v0.38 to remove adapters and low-quality sequences. The quality of trimmed sequences was checked using FastQC-v0.11.7. The trimmed data were then analyzed using tophat/2.1.1 to map read to the reference genome of the doubled monoploid S. tuberosum (DM), and read counts of potato genes were obtained using Cuffdiff of the cufflinks/2.2.1. The gene read counts were converted to tab-delimited text file using the Perl script parse_cuffdiff_ readgroup.pl (Dr. Qi Sun; Institute of Biotechnology at Cornell University). The differentially expressed genes (DEGs, adjusted p-value <0.05) between treatments at 0 and 6 hours were analyzed using the R package edgeR-v3.24.3. Gene ontology (GO) enrichment analysis was performed on the DEGs using Fisher's exact test using OmicsBox software. The GO terms of potato genes were retrieved from BioMart at EnsemblPlants.

The expression profiling revealed that StPep1 treatment led to 392 up-regulated genes and 373 down-regulated genes (≥2 fold log change at 6 hpi, q<0.05).

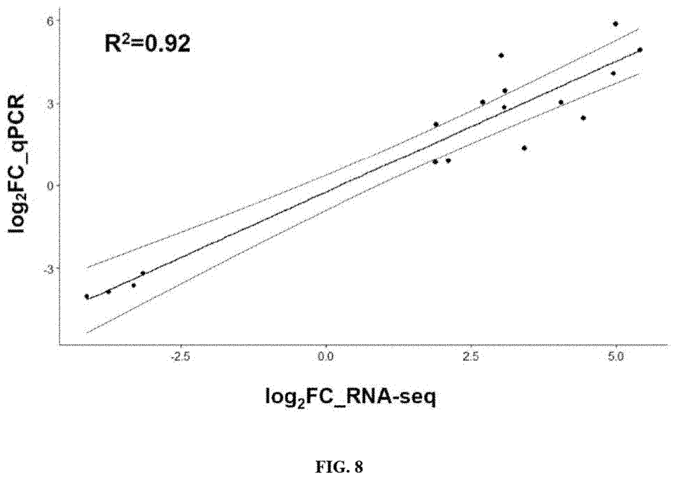

The results of RNA-seq were also validated by qRT-PCR analysis of a subset of these differentially expressed genes (FIG. 8).

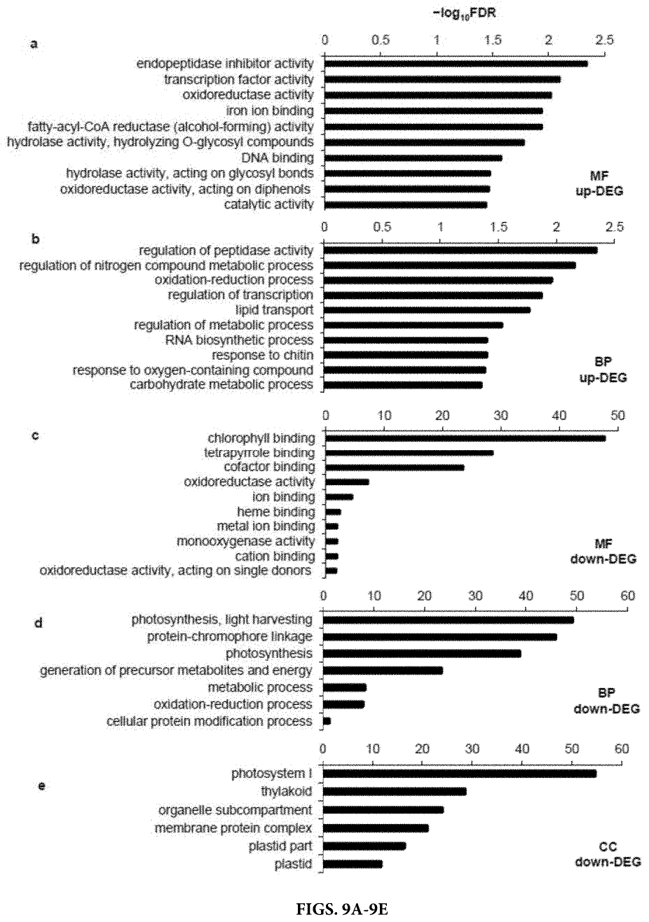

Over-representation of Gene Ontology (GO) terms was then investigated in the differentially expressed genes (FIG. 9). A total of 8 up-regulated genes that fell into the highly enriched GO terms, endopeptidase inhibitor activity and transcription factor activity was selected to develop StPep1-induced marker genes in potato roots.

These 8 genes were: DMG400015290; DMG40024067; DMG40016270; DMG400000380; DMG40008223; DMG400017787; DMG400019294; and DMG400015342.

Potato plants by root soaking was treated with either water or 100 nM StPep1 and measured the expression of these 8 genes by qRT PCR in both roots and leaves at 6 hours post-treatment. In the leaves, the expression of these eight genes varied. However, in the roots, seven of the eight genes were up-regulated by StPep1 treatment and, thus, could serve as marker genes for potato roots treated with StPep1.

These 7 genes were: DMG400015290; DMG40024067; DMG40016270; DMG400000380; DMG40008223; DMG400017787; and DMG400019294.

### Example 5

### Analysis of StPep1 Marker Gene Expression in Potato Roots

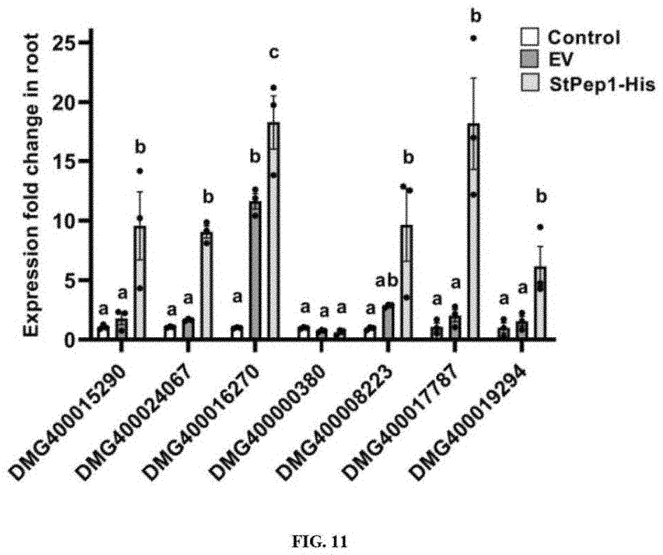

The instant example was performed in order to determine if the exemplary Bacillus-secreting StPep1 engineered bacterial strain affected expression on the seven marker genes identified for StPep1 in potato roots. Roots were treated with water or the Bacillus cultures (empty vector control or Bacillus-secreting His-tagged StPep1) for 6 hours. The Bacillus-secreting His-tagged StPep1 significantly increased expression of five of the seven StPep1-marker genes compared to the controls (FIG. 11). This indicates that the Bacillus-secreting StPep1 can trigger root gene expression similar to StPep1 in solution. When combined with the induced nematode resistance data, the data suggest that the Bacillus is secreting an active plant defense elicitor.

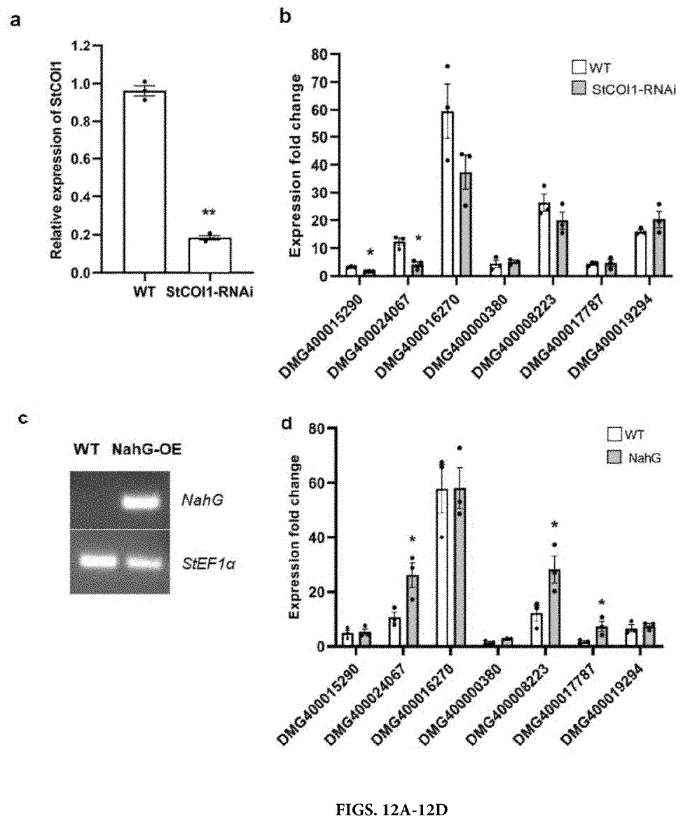

The JA receptor COI1 is required to some extent for AtPep1 immune responses in A. thaliana. Therefore, 7 StPep1-responsive genes were evaluated to determine if they were dependent on JA perception in potato roots by using potato plants knocked down in the JA receptor StCOI1 (referred to StCOI1-RNAi) (FIG. 12A and FIG. 12B). The fold change in gene expression at 6 hours between the mock treatment and StPep1 treatment in the WT and StCOI1-RNAi roots was measured. IT was found gene induction was reduced in the StCOI1-RNAi plants for 2 of the 7 marker genes tested (FIG. 12B). The data indicate some gene expression outputs in roots requires the JA receptor for StPep1-mediated signaling.

### Example 6

### Evaluation of StPep1-Responsive Genes and Salicylic Acid

Because A. thaliana PROPEP genes are induced by methyl salicylate treatments, the expression of the StPep1-responsive genes in roots were tested to determine if they required salicylic acid (SA). The roots of the wild-type potatoes and potatoes expressing the bacterial NahG gene (FIG. 12C) were treated with water or StPep1 for 6 hours, and the expression of the 7 StPep1-responsive genes was measured in the roots. The fold change in gene expression at 6 hrs was measured between the mock treatment and StPep1 treatment in the WT and NahG roots. The results showed that expressions of 3 of the 7 marker genes were significantly higher in NahG plants compared with WT (FIG. 12D).

1. A bacterial strain modified for secretion of a plant defense elicitor peptide, said bacterial strain comprising an exogenous sequence encoding the plant defense elicitor peptide.

2. The bacterial strain of claim 1, wherein the bacterial strain is a Bacillus bacterial strain.

3. The bacterial strain of claim 2, wherein the Bacillus bacterial strain is Bacillus subtilis.

4. The bacterial strain of claim 1, wherein the plant defense elicitor peptide is StPep1.

5. The bacterial strain of claim 1, wherein the plant defense elicitor peptide is SlPep6.

6. The bacterial strain of claim 1, wherein the plant defense elicitor peptide is AtPep1.

7. The bacterial strain of claim 1, wherein the exogenous sequence encoding the plant defense elicitor peptide is a sequence having at least 95% sequence identity to SEQ ID NO: 1 (ATERRGRPPSRPKVGSGPPPQNN).

8. The bacterial strain of claim 1, wherein the exogenous sequence encoding the plant defense elicitor peptide is a sequence having at least 95% sequence identity to SEQ ID NO: 2 (ATDRRGRPPSRPKVGSGPPPQNN).

9. The bacterial strain of claim 1, wherein the exogenous sequence encoding the plant defense elicitor peptide is a sequence having at least 95% sequence identity to SEQ ID NO: 3: ATKVKAKQRGKEKVSSGRPGQHN.

10. The bacterial strain of claim 1, wherein the modified strain is configured to colonize one or more roots of a plant for at least 10 days.

11. A method of controlling root galling in a plant, said method comprising the step of administering a composition comprising a bacterial strain modified for secretion of a plant defense elicitor peptide to the plant,
wherein the bacterial strain comprises an exogenous sequence encoding the plant defense elicitor peptide, and
wherein administration of the composition to the plant controls root galling in the plant.

12. The method of claim 11, wherein the plant is a dicot plant selected from the group consisting of a potato plant, a tomato plant, and an Arabidopsis plant.

13. The method of claim 11, wherein the plant is a potato plant.

14. The method of claim 11, wherein the composition comprises a liquid, wherein the liquid is water.

15. The method of claim 11, wherein the administration is applied via irrigation.

16. The method of claim 11, wherein the bacterial strain is a Bacillus bacterial strain.

17. The method of claim 16, wherein the Bacillus bacterial strain is Bacillus subtilis.

18. The method of claim 11, wherein the plant defense elicitor peptide is selected from the group consisting of StPep1, SlPep6, and AtPep1.

19. The method of claim 11, wherein the root galling is caused by a root-knot nematode, wherein the root-knot nematode is a Meloidogyne nematode, wherein the Meloidogyne nematode is Meloidogyne chitwoodi, Meloidogyne incognita, or both.

20. The method of claim 11, wherein administering the composition to the plant is performed by treating the soil near or adjacent to one or more roots of the plant.
